(19) United States
(12) Patent Application Publication
(10) Pub. No.: US 2025/0090199 A1
(43) Pub. Date: Mar. 20, 2025

(54) Cupid

(71) Applicant: Natalia Fields, Dallas, TX (US)

(72) Inventor: Natalia Fields, Dallas, TX (US)

(21) Appl. No.: 18/368,055

(22) Filed: Sep. 14, 2023

(51) Int. Cl. A61B 17/43

(52) U.S. Cl. CPC A61B 17/43

(57) ABSTRACT

A fertility aid device includes a cup defining a chamber to receive and store sperm and configured to be inserted inside a vagina of a woman to deliver the sperm inside the vagina to a cervix. The device also includes a string attached to the cup and extending outwardly of the cup to facilitate a removal of the cup from the vagina.

### BACKGROUND

Couples and women often have difficulty with fertility. Consequently, there is a need for a system and method which assists with fertility.

### SUMMARY

One aspect of this disclosure relates to a fertility aid device. The fertility aid device includes a cup defining a chamber to receive and store sperm. The cup is configured to be inserted inside a vagina of a woman to deliver the sperm near a cervix to aid in conception. The fertility aid device also includes a string attached to the cup and extending outwardly of the cup to facilitate a removal of the cup from the vagina.

In some additional, alternative, or selectively cumulative embodiments, the fertility aid device further comprises a handle attached to the string to facilitate a holding of the fertility aid device by a user.

In some additional, alternative, or selectively cumulative embodiments, the handle includes a spherical shape.

In some additional, alternative, or selectively cumulative embodiments, the handle includes a heart shape.

In some additional, alternative, or selectively cumulative embodiments, the cup includes a flexible portion to enable a manipulation of the cup to facilitate the insertion of the cup inside the vagina.

In some additional, alternative, or selectively cumulative embodiments, the flexible portion includes a top of the cup adapted to be pinched such that at an opening of the cup defines a figure 8 shape.

In some additional, alternative, or selectively cumulative embodiments, the cup and the string are integrally formed with each other.

In some additional, alternative, or selectively cumulative embodiments, the cup is made of a medical grade silicone.

One aspect of this disclosure relates to a method for providing a fertility aid to a woman. The method comprises providing a fertility aid device. The fertility aid device includes a cup to receive and store sperm and a string attached to the cup to facilitate an insertion and removal of the cup from a vagina of the woman. The method further includes collecting sperm inside the cup of the fertility aid device, and inserting the cup having the sperm inside the vagina and positioning the cup proximate to a cervix inside the vagina to deliver the sperm near the cervix to aid in conception.

In some additional, alternative, or selectively cumulative embodiments, the method further comprises manipulating the cup to enable the insertion of the cup inside the vagina.

In some additional, alternative, or selectively cumulative embodiments, manipulating the cup includes pinching a top of the cup such that an opening of cup defines a figure 8 shape.

In some additional, alternative, or selectively cumulative embodiments, manipulating the cup includes a folding the cup.

In some additional, alternative, or selectively cumulative embodiments, the method further comprises removing the cup from the vagina upon elapse of a predefined time period from the insertion of the cup inside the vagina.

In some additional, alternative, or selectively cumulative embodiments, the cup is removed from the vagina by pulling the string.

### BRIEF DESCRIPTION OF THE ACCOMPANYING DRAWINGS

Having thus described example embodiments of the present disclosure in general terms, reference will now be made to the accompanying drawings, which are not necessarily drawn to scale, and wherein:

FIG. 1 is a bottom perspective view of a fertility aid device, in accordance with an embodiment of the disclosure;

FIG. 2 is a side view of the fertility aid device of FIG. 1, in accordance with an embodiment of the disclosure;

FIG. 3 is a front view of the fertility aid device of FIG. 1, in accordance with an embodiment of the disclosure;

FIG. 4 is a sectional view of the fertility aid device of FIG. 3 along a section line A-A, in accordance with an embodiment of the disclosure;

FIG. 5 is a top view of the fertility aid device of FIG. 1, in accordance with an embodiment of the disclosure; and

FIG. 6 is an enlarged view of a handle of the fertility aid device of FIG. 1, in accordance with an embodiment of the disclosure.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details. In other instances, apparatus and methods are shown in block diagram form only in order to avoid obscuring the present disclosure.

Example embodiments are described below with reference to the accompanying drawings. Unless otherwise expressly stated in the drawings, the sizes, positions, etc., of components, features, elements, etc., as well as any distances therebetween, are not necessarily to scale, and may be disproportionate and/or exaggerated for clarity.

The terminology used herein is for the purpose of describing example embodiments only and is not intended to be limiting. As used herein, the singular forms “a,” “an” and “the” are intended to include the plural forms as well, unless the context clearly indicates otherwise. It should be recognized that the terms “comprise,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise specified, a range of values, when recited, includes both the upper and lower limits of the range, as well as any sub-ranges therebetween. Unless indicated otherwise, terms such as “first,” “second,” etc., are only used to distinguish one element from another. For example, one element could be termed a “first element” and similarly, another element could be termed a “second element,” or vice versa. The section headings used herein are for organizational purposes only and are not to be construed as limiting the subject matter described.

Unless indicated otherwise, the terms “about,” “thereabout,” “substantially,” etc., mean that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art.

Spatially relative terms, such as “right,” left,” “below,” “beneath,” “lower,” “above,” and “upper,” and the like, may be used herein for ease of description to describe one element's or feature's relationship to another element or feature, as illustrated in the drawings. It should be recognized that the spatially relative terms are intended to encompass different orientations in addition to the orientation depicted in the figures. For example, if an object in the figures is turned over, elements described as “below” or “beneath” other elements or features would then be oriented “above” the other elements or features. Thus, the term “below” can, for example, encompass both an orientation of above and below. An object may be otherwise oriented (e.g., rotated 90 degrees or at other orientations) and the spatially relative descriptors used herein may be interpreted accordingly.

Unless clearly indicated otherwise, all connections and all operative connections may be direct or indirect. Similarly, unless clearly indicated otherwise, all connections and all operative connections may be rigid or non-rigid.

Like numbers refer to like elements throughout. Thus, the same or similar numbers may be described with reference to other drawings even if they are neither mentioned nor described in the corresponding drawing. Also, even elements that are not denoted by reference numbers may be described with reference to other drawings.

Many different forms and embodiments are possible without deviating from the spirit and teachings of this disclosure and so this disclosure should not be construed as limited to the example embodiments set forth herein. Rather, these example embodiments are provided so that this disclosure will be thorough and complete, and will convey the scope of the disclosure to those skilled in the art.

Reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced items. Moreover, various features are described which may be exhibited by some embodiments and not by others. Similarly, various requirements are described which may be requirements for some embodiments but not for other embodiments.

Some embodiments of the present disclosure will now be described more fully hereinafter with reference to the accompanying drawings, in which some, but not all, embodiments of the disclosure are shown. Indeed, various embodiments of the disclosure may be embodied in many different forms and should not be construed as limited to the embodiments set forth herein; rather, these embodiments are provided so that this disclosure will satisfy applicable legal requirements. Like reference numerals refer to like elements throughout. The use of any term should not be taken to limit the spirit and scope of embodiments of the present disclosure.

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present disclosure. Further, it is to be understood that the phraseology and terminology employed herein are for the purpose of the description and should not be regarded as limiting. Any heading utilized within this description is for convenience only and has no legal or limiting effect.

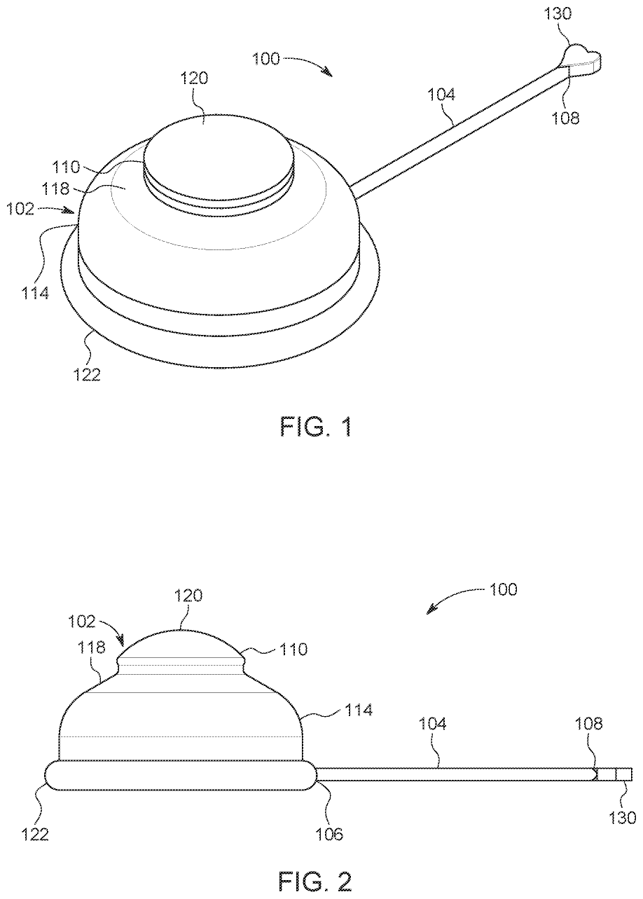

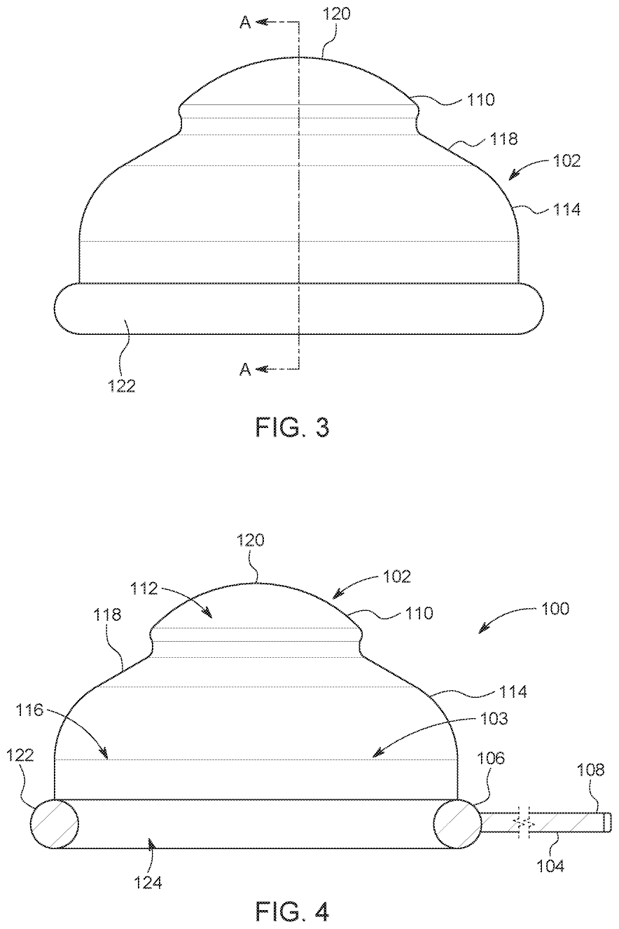

Referring to FIGS. 1 to 6, a fertility aid device 100 is shown, according to an example embodiment of the disclosure. As shown, the fertility aid device 100 (hereinafter simply referred to device 100) includes a cup 102 defining a chamber 103 to receive and hold a substance, for example, sperm, and a string 104 attached to the cup 102 and extending outwardly from the cup 102. The string 104 includes a first end 106 attached to the cup 102 and a second end 108 arranged distally from the cup 102. The string 104 facilitates a removal of the cup 102 from a vagina of a woman.

As shown in FIGS. 2, 3, and 4 the cup 102 includes a base portion 110 defining a lower chamber 112 (shown in FIG. 4) to receive and store the sperm and a top portion 114 extending upwardly from the base portion 110 and defining an upper chamber 116 (shown in FIG. 4) of the cup 102. As shown, a diameter of the base portion 110 is smaller than a diameter of the top portion 114 defining a step 118 therebetween. Moreover, as shown on FIGS. 1-4, a base 120 of the base portion 110 includes a substantially convex shape. Further, the cup 102 includes a lip 122 arranged at a free edge of the top portion 114 of the cup 102 and defining an opening 124 of the chamber 103. It may be appreciated that the upper chamber 116 and the lower chamber 112 together define the chamber 103 of the cup 102.

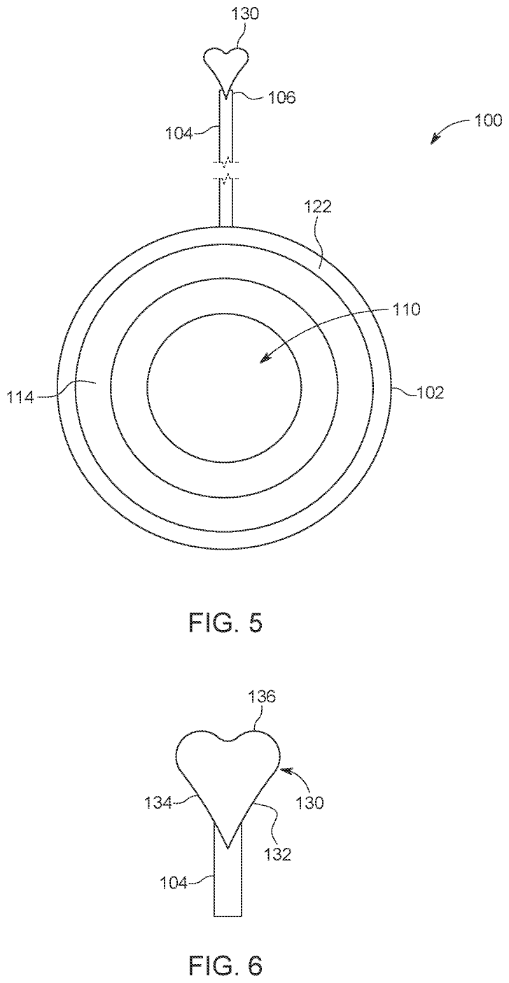

In some embodiments, as shown in FIGS. 1 and 5, the device 100 may include a handle 130 arranged at the second end 108 of the string 104 and attached to the string 104. The handle 130 provides a grip portion for a user to hold the device 100 and enables an insertion of the cup 102 inside the vagina and removal of the cup 102 from the vagina. Also, the handle 130 facilitates a collection of the sperm inside the cup 102 without contacting the cup 102. The handle 130 may include any shape or size known in the art. In the illustrated embodiment, as shown in FIG. 6, the handle 130 includes a heart shape having two longitudinal edges 132, 134, each extending obliquely and outwardly from the string 104 at an angle. In some embodiments, the angle may be between 25 degrees to 35 degrees. In an embodiment, the angle may be 32 degrees. Further, the handle 130 includes a lateral edge 136 connecting the two longitudinal edges 132, 134. As shown, the lateral edge 136 includes a wavy shape with a trough of the wave arranged substantially centrally to the lateral edge 136. Accordingly, the lateral edge 136 includes two arcuate portions connected to each other at a center of the lateral edge 136. In an embodiment, each arcuate portion includes a predefined radius. In some embodiments, the handle 130 may include a spherical shape instead of a heart shape.

In some embodiments, the cup 102, the string 104, and the handle 130 are integrally formed. Alternatively, the cup 102 and the string 104 may be engaged rigidly or removably with each other. In some embodiments, the device 100 may include any bio-safe material known in the art. In one embodiment, the device 100 may include rubber, plastics, or a combination thereof. In one embodiment, the device 100 is a class 2 medical device. In one embodiment, the device 100 is made of medical grade silicone.

A method for aiding in a fertility of a woman using the device 100 is now described. The method includes a step at which a user or an operator collects the sperm inside the cup 102. The sperm may comprise live sperm, thawed frozen sperm, etc. The amount of sperm collected inside the cup 102 depends upon a size of the cup 102. As the sperm are collected/placed directly inside the cup 102 rather than using various transporting or intermediate equipment, a risk of spill and losses of the sperm in minimized. Further, placing/collecting the sperm directly inside the cup 102 reduces the complexity, and simplifies the process.

In some optional embodiments, the operator before collecting the sperm inside the cup 102, the operator or the user sanitizes the device 100. For so doing, in an example embodiment, the device 100 is positioned inside boiling water for a predefined time, for example, at least five minutes. Although, the sanitization of the device 100 using the boiling water is contemplated, it may be appreciated that the device 100 may be sanitized any other method known in the art. The sanitization of the device 100 sterilizes the device 100 and helps in killing any bacteria located on the device 100.

Upon sanitizing the device 100, in some optional embodiments, the operator washes the device 100. Optionally, the operator may wash his or her hands and wear medical grade gloves. After washing or sterilizing, the device 100 is ready for use.

After depositing the sperm into the cup 102, the method includes a step at which the operator may manipulate the cup 102 for suitable insertion inside the vagina of the woman. In some embodiments, the operator may manipulate the cup 102 in different forms depending upon the shape and size of the cup 102 to enable/facilitate the insertion of the cup 102 inside the vagina. For example, the operator may pinch a top of the cup 100 such that the opening 124 takes either an oval shape or a figure 8 shape. In some embodiments, the manipulation of the cup 102 includes folding the cup 102 in a manner so that it may be subsequently inserted.

Subsequent to the manipulation of the cup 102, the operator or user inserts the cup 102 inside the vagina. Cup 102 is inserted in such a way as to not spill the sperm outside the cup 102 during insertion. In one embodiment, the woman lays down facing upward, and the cup 102 is inserted inside the vagina such that the opening 124 i.e., open end of the cup 102 is facing upward. It may be appreciated that the user or the operator pushes the cup 102 inside the vagina to the cervix until the cup 102 does not move further.

Upon insertion of the cup 102 inside the vagina and up and into the cervix, the cup 102 is left inside the vagina for a predefined period of time. This allows the sperm to exit the cup 102. In some embodiments, the cup 102 is removed from the vagina after the elapse of the predefined time period, for example, six hours from the insertion. This help in delivering the sperm to the cervix to aid in conception. For removal of the cup 102 from the vagina, the user pulls the string 104 by holding the string 104 via the handle 130. Accordingly, a length of the string 104 is selected such that a portion of the string 104 and/or the handle 130 remains outside the vagina when the cup 102 is completely inserted inside the vagina. It may be appreciated that the user or the operator washes hands before pulling the string 104 to remove the cup 102 from the vagina. The process can be repeated several times until fertility is achieved.

In this manner, the cup 102 acts as both a sperm container and a conception device. This is an advantage compared to prior devices which needed a separate cup which is syringed or suctioned out and then delivered to the conception device. As such, the instant device 100 provides a single device which acts as both the collection and delivery device. Moreover, the device 100 is a relatively inexpensive that can be used to increase fertility. Also, the device 100 is an over the counter device and may be used by the couples/women at home. Further, the device 100 may be used during the ovulatory phase of the menstrual cycle.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

What is claimed is:

1. A fertility aid device, comprising:
a cup defining a chamber to receive and store sperm and configured to be inserted inside a vagina of a woman to deliver the sperm near a cervix; and
a string attached to the cup and extending outwardly of the cup to facilitate a removal of the cup from the vagina.

2. The fertility aid device of claim 1 further comprising a handle attached to the string to facilitate a holding of the fertility aid device by a user.

3. The fertility aid device of claim 2, wherein the handle includes a heart shape.

4. The fertility aid device of claim 1, wherein the cup includes a flexible portion to enable a manipulation of the cup to facilitate the insertion of the cup inside the vagina.

5. The fertility aid device of claim 4, wherein the flexible portion a top of the cup adapted to be pinched such that an opening of the cup defines a figure 8 shape.

6. The fertility aid device of claim 1, wherein the cup and the string are integrally formed with each other.

7. The fertility aid device of claim 1, wherein the cup is made of a medical grade silicone.

8. A method for providing a fertility aid to a woman, the method comprising:
providing a fertility aid device including a cup to receive and store sperm and a string attached to the cup to facilitate an insertion and removal of the cup from a vagina of the woman;
collecting sperm inside the cup of the fertility aid device; and
inserting the cup having the sperm inside the vagina and positioning the cup proximate to a cervix inside the vagina to deliver the sperm near the cervix.

9. The method of claim 8 further comprising manipulating the cup to enable the insertion of the cup inside the vagina.

10. The method of claim 9, wherein manipulating the cup includes pinching a top of the cup such that an opening of the cup defines a figure 8 shape.

11. The method of claim 9, wherein manipulating the cup includes a folding the cup.

12. The method of claim 8 further comprising removing the cup from the vagina upon elapse of a predefined time period from the insertion of the cup inside the vagina.

13. The method of claim 12, wherein the cup is removed from the vagina by pulling the string.
